(19) United States
(12) Patent Application Publication
(10) Pub. No.: US 2002/0125184 A1
(43) Pub. Date: Sep. 12, 2002

(54) Treatment of physiological liquids of organism

(76) Inventors: Hans-Dietrich Polaschegg, Koaatenberg (AT); James Brady, Riverdale, NY (US); Ilya Zborovsky, Dix Hills, NY (US)

Correspondence Address: Ilya Zborovsky, 6 Schoolhouse Way, Dix Hills, NY, 11746, US

(21) Appl. No.: 10/029,865

(22) Filed: Dec. 31, 2001

Related U.S. Application Data
(63) Continuation of application No. 09/597,670, filed on Jun. 21, 2000.

(51) Int. Cl. B01D 15/00

(52) U.S. Cl. 210/263

(57) ABSTRACT

A method of treatment of physiological liquids of organism includes providing a housing with inlet and outlet for physiological liquid so that physiological liquid enters an interior of the housing through the inlet and exits the same through the outlet, arranging a body of treatment material accommodated in the interior of the housing so that the physiological liquid which passes through the housing from the inlet to the outlet is treated by the treatment material, uniformly distributing the physiological liquid by a body of particulate material located upstream the body of treatment material between the inlet and the body of treatment material so as to provide a uniform distribution of the physiological liquid after it enters the housing and before it enters the body of treatment material; and arranging a further body of particulate material located downstream of the body of treatment material between the body of treatment material and the outlet.

### CROSS REFERENCE TO A RELATED APPLICATION

This application is a continuation of application Ser. No. 09/597,670.

### BACKGROUND OF THE INVENTION

The present invention relates to method of treatment of physiological liquids of organism.

More particularly, it relates to methods which are used for dialysis or filtration treatment.

Some methods of the above mentioned general type are implemented in cartridges which are formed as self-contained, mobile and compact units filled with hollow fibers for performing dialysis or filtration treatments of physiological liquids of organism. Such a dialyzer cartridge is described, e.g, in U.S. Pat. No. 5,700,372. A cartridge usually has a housing provided with an upstream inlet and a downstream outlet. The inlet is usually formed as an narrow passage through which a physiological liquid is admitted into the interior of the cartridge and then spreads laterally to pass through the fibers for physiological liquid treatment. A substantial disadvantage of such cartridges is that the physiological liquid is not uniformly distributed over the flow cross-section of the interior of the cartridge since it enters the cartridge as a narrow stream and then expands laterally. This feature has been measured on actual cartridges by, e.g., “Ronco C, Fabris A, Feriani M, Chiaramonte S, Brendolan A, Emiliani G, La Greca G. Technical and clinical evaluation of a new synthetic low flux polysulphon membrane for hemodialysis. Int J Artif Organs 1989;12:450-60”. The non uniformity of the flow of a physiological liquid through the material for treatment naturally affects the efficiency and quality of treatment of the physiological liquid in the cartridge.

### SUMMARY OF THE INVENTION

Accordingly, it is an object of the present invention to provide a method of treatment of physiological liquids of organism, which avoids the disadvantages of the prior art.

In keeping with these objects and with others which will become apparent hereinafter, one feature of present invention resides, briefly stated, in a method of treatment of physiological liquids of organism which includes the steps of providing a housing with upstream inlet means and downstream outlet means for a physiological liquid; arranging a body for treatment of the physiological liquid and including a plurality of linearly elongated mass transfer elements; uniformly distributing a flow of the physiological liquid after its passage through said inlet means by a body of a particulate material located upstream of said body for treatment; and also uniformly distributing the flow of the physiological liquid by a body of particulate material located downstream of said body of treatment.

When the method is performed in accordance with the present invention, each body of the particulate material can act as a flow diffuser which diffuses a flow of the physiological liquid of organism immediately after its entry in the cartridge, with minimal dead space. Since two bodies of particulate material are located at both sides of the treatment body, the device becomes symmetrical. It can be connected to a patient by any end, and no special coding of its ends is needed.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view schematically showing a device for implementing a method of treatment physiological liquids of organism in accordance with the present invention;

FIGS. 2a-2d are views showing elements for treatment of physiological liquids, accommodated in the device implementing a method in accordance with the present invention;

FIGS. 3a-3c are views showing elements of a body of particulate material accommodated in the cartridge implementing a method in accordance with the present invention.

### DESCRIPTION OF PREFERRED EMBODIMENTS

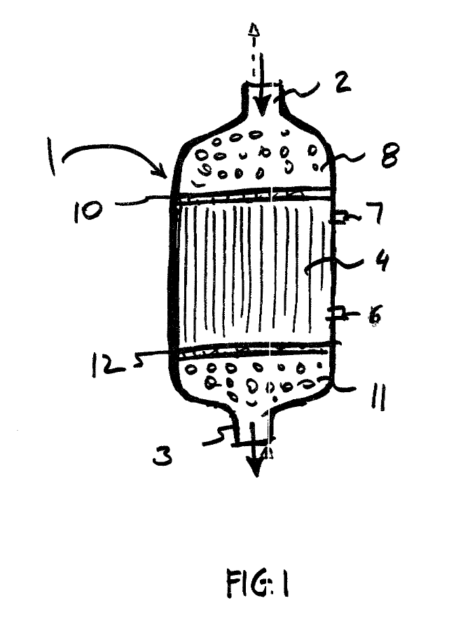

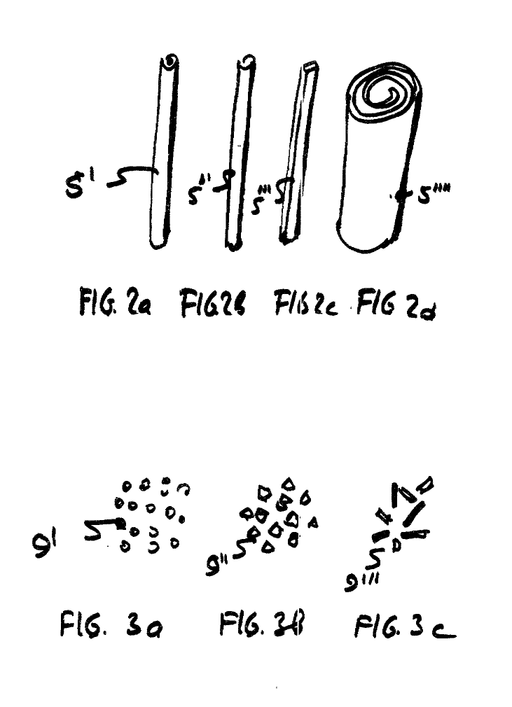

A method of treatment of physiological liquids of organism, such as blood, plasma, peritoneal liquid, etc., is performed in a device which is formed for example as a cartridge. It has a housing which is identified as a whole with reference numeral 1 and provided with an inlet 2 and an outlet 3 for the physiological liquid as can be seen from FIG. 1. The housing 1 is partially filled with a body of a material which provides treatment of the physiological liquid and is identified with reference numeral 4. The material 4 is preferably composed of linearly elongated mass transfer elements through which the physiological liquid passes and is subjected to a corresponding treatment. The material for treatment can be in form of, for example, hollow fibers, solid fibers, plate like elements, spiral elements, etc. as specifically shown in FIGS. 2a, 2b, 2c, 2d, and 2e and identified reference numerals 5′, 5″, 5′″, 5″″.

The treatment material 4 can be formed so as to perform a dialysis, for which purpose the housing can be provided with a dialysis fluid inlet 6, a dialysis fluid outlet 7. While the physiological liquid flows in direction from the inlet 2 to the outlet 3 through the material 4, the dialysis liquid will flow in a counter current from the inlet 6 to the outlet 7 of the dialysis fluid. The treatment material 4 is formed in this case so as to perform dialysis and is composed of hollow fibers with semipermeable walls made from, e.g., polysufone, polyethersulfon, cuprophane.

The dialysis process is performed in the following manner: Physiological fluid is enters the cartridge at entrance 2, passes the flow diffusing material and enters the lumen of the dialysis fibers. It leaves the cartridge at 3. Simultaneously, dialysate flows through the cartridge at the outside of the dialysis fibers in a countercurrent mode entering the cartridge at 6 and leaving the cartridge at 7.

The treatment material 4 can be used for performing filtration of the physiological liquid. In this case the material is composed of hollow fibers with semipermeable walls preferably of high water permeability made from, e.g., polysufone, polyethersulfon. The filtration is performed in the following manner. Physiological fluid is enters the cartridge at entrance 2, passes the flow diffusing material and enters the lumen of the dialysis fibers. It leaves the cartridge at 3. A pressure differential is built up between the inside and the outside of the fibers causing ultrafiltration of fluid from the inside to the outside. This filtrate leaves the cartridge through connections 6 and/or 7.

The treatment material 4 can also perform adsorption of toxins from the physiological liquid. In this case the material is formed as hollow fibers, solid fibers, plate like elements or spiral wound elements. In case of hollow fibers the physical design of the cartridge is similar to the cartridge used for hemodialysis or hemofiltration with the difference that the fibers are made from a non-porous material. Alternatively porous material can be used as well and a combination of adsorption and hemodialysis or hemofiltration, repsectively, can be performed. Hemodialysis or hemofiltration fibers are kept in place within a cartridge by potting material that encloses the fibers leaving the lumen of the fibers open. Any other not hollow material, e.g., fibers are preferably bundled and separated from the flow diffuser part by a mesh or screen allowing passage of the physiological fluid but not of the particles used for flow diffusion. The adsorption process is performed in the following manner: Physiological fluid passes the cartridge from entrance 2 to exit 3. Unwanted components in the physiological fluid are adsorbed on the surface of the adsorption material, e.g., the fiber or in pores of the said material. Materials suitable for this purpose are, e.g., polysulphone or PMMA or PAN for adsorption of beta-2-microglobulin or carbon fibers or fibers, hollow fibers, spiral wound foils coated with carbon or any other suitable material.

The material 4 can also be formed for performing ion exchange, e.g., for exchanging potassium against sodium. Any of the above mentioned physical forms can be used for this purpose. Ion exchange resins can be spun into fibers (Matsuda K, Oka T, Tani T, Hanasawa K, Yoshioka T, Aoki H, Endo Y, Ishii Y, Numa K, Kodama M. Experimental study on the adsorption of excess heparin with anion exchange resin fiber. Artif Organs 1989;13:504-7), filled into the lumen of capillary dialyzers or can be grafted on a carrier, e.g., a fiber or sheet material. The ion exchange is performed in the manner as decribed above.

In accordance with the present invention, the physiological liquid after entering the housing through the inlet 2 is uniformly distributed, before being treated, by a body of particulate material is located upstream of the body of treatment material 4, when considered in direction of flow of the physiological liquid. The body of the particulate material is identified as a whole with reference numeral 8. It is located between the inlet 2 for the physiological liquid and the body of treatment material 4 for treatment of the physiological liquid. When the body of the particulate material 8 is located in this way, the physiological liquid entering the cartridge through the inlet 2 is uniformly distributed over the cross-section of the cartridge upstream of the body of treatment material 4 and then passes through the linearly elongated mass transfer elements in a uniform fashion, which substantially increases quality, uniformity and efficiency of treatment of the physiological liquids.

The particulate material 8 can be formed by a plurality of polymer beads 9′, grains 9″, short fibers 9″ shown in FIGS. 3a, 3b, 3c or in other forms as well. In accordance with one embodiment of the present invention, the particulate material 8 performs exclusively the functions of uniform distribution of the flow of physiological liquids upstream of the treatment material 4. In accordance with another embodiment of the present invention, the particulate material 8 can also perform treatment functions or in other words acts in a certain effective way on the physiological liquid, in addition to its distribution over the cross-section of the cartridge.

In accordance with another embodiment of the present invention, the particulate material 8 can also provide adsorption of toxins from physiological liquid of organism, such as for example beta-2-microglobulin.

For this purpose, a suitable particular material 8 is, e.g., described in the patent WO9906098.

In addition, the particulate material 8 can also perform the functions of endotoxin adsorption. An appropriate material would be Polymixin-B or alternatively materials described in U.S.Pat. No.3,959,128.

The particulate material 8 can also remove lipids. For this purpose it can be composed of ,e.g., a material as described in EP0424698.

In the method in accordance with the present invention the body of the material for treatment 4 can be separated from the body of the particulate material 8 by a separating element to prevent intermixing of the materials. The separating element is identified with reference numeral 10 and can be composed for example of a thin mesh. The openings of the mesh are smaller at least than the particles of the particulate material 8 to prevent the particulate material 8 from falling into the body of the treatment material. Preferably, the openings of the mesh are also smaller than the elements of the treatment material 4. In case the treatment material 4 consists of hollow fibers the mesh is replaced by the potting material fixing the hollow fibers. The size of the particulate material in this case is larger than the opening of the hollow fiber. Alternatively an additional mesh can be put in front of the hollow fiber entrance.

In accordance with the present invention as shown in FIG. 4, another body of particulate material 11 is located downstream of the body of treatment material 4. The particulate material 11 can also be composed of beads, grains, short fibers, etc. The body of the particulate material 11 is separated from the body of treatment material 4 by another partition 12 which can also be formed as a corresponding mesh.

The material of the body 11 can be composed of elements which just provide uniform distribution of the physiological liquid over the cross-section of the cartridge. Also, the elements can additionally release medications, vitamins, hormones, electrolytes, etc. into the physiological liquid of organism which has passed through the treatment body 4. Such substances are known in the art. The particulate material 11 can also additionally remove toxins from the physiological liquid.

Since the device has two bodies of particulate material 8 and 11 at both ends of the body of treatment material, the cartridge becomes symmetrical. In known cartridges the ends are usually color-coded to provide the connection of the corresponding ends to a patient. In the inventive cartridge it is no longer needed, since both ends with their bodies of particulate materials 8 and 11 are identical in their structure and function. Therefore, the uniform distribution of the physiological liquid can be performed with the inlet located upstream of the body of the treatment material, or in an upside down position of the device when the inlet is located downstream of the body of the treatment material.

It will be understood that each of the elements described above, or two or more together, may also find a useful application in other types of constructions differing from the types described above.

While the invention has been illustrated and described as embodied in a cartridge for treatment of physiological liquids of organism, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of treatment of physiological liquids of organism, comprising the steps of providing a housing with inlet means and outlet means for physiological liquids so that physiological liquid enters an interior of said housing through said inlet means and exits the same through said outlet means; arranging a body composed of treatment material which is formed to provides treatment of a physiological liquids of organism and is accommodated in the interior of said housing so that the physiological liquid which passes through said housing from said inlet means to said outlet means through said body of said treatment material is treated by said treatment material; uniformly distributing the physiological liquid of organism before it enters said body of said treatment material by a body of particulate material located upstream said body of treatment material between said inlet means and said body of treatment material so as to provide a uniform distribution of the physiological liquid said housing and before said body of treatment material; and providing a uniform distribution of the physiological liquid of organism by a further body of particulate material located downstream of said body of treatment material between said body of treatment material and said outlet means, so that when the device is turned upside down and said outlet means become said inlet means, said further body of particulate material is located upstream of said body of treatment material between said outlet means which became said inlet means on the one hand and said body of treatment material of the other hand to again provide a uniform distribution of physiological liquid before it enters said body of treatment material.

2. A method as defined in claim 1; and further comprising using as said treatment material a material selected from the group consisting of hollow fibers, solid fibers, material plates, and a wound structure.

3. A method as defined in claim 1; and further comprising using as said particulate material a material selected from the group consisting of beads, grains, and short fibers.

4. A method as defined in claim 1; and further comprising using as said treatment material a material formed so a to perform a process selected from the group consisting of a dialysis, a filtration, an adsorption and an ion exchange.

5 A method as defined in claim 1; and further comprising using as said particular material a material which is formed so as to provide exclusively the function of uniform distribution of the physiological liquid.

6. A method as defined in claim 1; and further comprising using as said particulate material a material which in addition to providing a uniform flow distribution of the physiological liquid, provides an additional function.

7. A method as defined in claim 1; and further comprising using as the particulate material a material which is formed so as to perform treatment of the physiological liquid by adsorption of toxins.

8. A method as defined in claim 1; and further comprising preventing intermixing of said particular material with said treatment material.

9. A method as defined in claim 8, wherein said intermixing preventing includes using a mesh means having an opening size which is smaller than a size of particles of said particulate material.

10. A method as defined in claim 1; and further comprising using as said body of particulate material a material which is formed so as to release at least one of medications into the physiological liquid of organism selected from the group consisting of hormones, vitamins, and electrolytes.

11. A method as defined in claim 1; and further comprising separating said body of treatment material from said further particulate material.

12. A method as defined in claim 1; and further comprising using as said bodies of particulate material which are identical and symmetrical relative to said body of treatment material.
